(19) United States
(12) Patent Application Publication
(10) Pub. No.: US 2026/0185008 A1
(43) Pub. Date: Jul. 2, 2026

(54) OLEFINS TO FUEL IN A SINGLE-STAGE OLIGOMERIZATION PROCESS

(71) Applicant: UOP LLC, Rosemont, IL (US)

(72) Inventors: Manuela Serban, Northbrook, IL (US); Debanjan Chakrabarti, Arlington Heights, IL (US); Nickolas Kapaun, Chicago, IL (US); Jeannie Mee Blommel, Oregon, WI (US); Kyle Krynski, Chicago, IL (US); Wei Pan, Hoffman Estates, IL (US); Deng-Yang Jan, Elk Grove Village, IL (US)

(21) Appl. No.: 19/367,471

(22) Filed: Oct. 23, 2025

Related U.S. Application Data
(60) Provisional application No. 63/739,097, filed on Dec. 26, 2024.

(51) Int. Cl. C10L 1/04; C07C 2/08

(52) U.S. Cl. CPC C10L 1/04; C07C 2/08

(57) ABSTRACT

This disclosure describes a process for producing a sustainable aviation fuel including oligomerizing a charge olefin stream over a single oligomerization catalyst to produce an oligomerized stream with greater than about 50% and less than about 99.5% olefins conversion per pass in the charge stream.

### FIELD

The field is the conversion of olefins to aviation fuel. The field particularly relates to a single-stage oligomerization process for creating aviation fuel.

### BACKGROUND

There is a strong push to reduce carbon emissions from the aviation sector which can, in part, be achieved by using Sustainable Aviation Fuel (SAF) instead of conventional (fossil) jet fuel. SAF can be generated by many methods. One method is oligomerization of C₂-C₈ range olefins produced from ethanol, methanol or CO₂, followed by hydrogenation. The base feedstock for this route is a mixture of C₂-C₈ olefins which must have zero or minimum lifecycle CO₂ footprint for subsequent production of SAF.

Molecular sieves such as microporous crystalline zeolite and non-zeolitic catalysts, particularly silicoaluminophosphates (SAPO), are known to promote the conversion of oxygenates such as methanol to light olefins. The highly efficient Methanol to Olefins (MTO) process may convert oxygenates to light olefins and was typically considered for plastics production. Light olefins produced from the MTO process are highly concentrated in ethylene and propylene and also contain significant concentrations of butenes, pentenes, and hexenes. When methanol derived from low carbon intensity feedstocks such as carbon dioxide or municipal solid waste is fed to an MTO unit, renewable light olefins are produced.

Ethylene can be dimerized and oligomerized into olefins such as C₄, C₆ and C₈ olefins. Propylene can be dimerized and oligomerized into olefins such as C₆, C₉ and C₁₂ olefins. Ethylene and propylene can be co-oligomerized into olefins such as C₅ and C₇ olefins. Olefin oligomerization is an exothermic process that can oligomerize smaller olefins into larger olefins. More specifically, it can convert olefins including oligomerized olefins into a distillate including jet fuel and diesel range products. The oligomerized distillate can be saturated for use as transportation fuels.

Typically, oligomerization of light olefins to heavier olefins is accomplished in a two-stage oligomerization reactor. The oligomerization reaction takes place predominantly in the liquid phase or in a mixed liquid and gas phase at a WHSV of 0.5 to 10 hr⁻¹ on an olefin basis in contact with a zeolite catalyst. Typically, 10-50 wt % ethylene in the olefin stream converts to higher olefins. The second-stage oligomerization reactor may be in downstream communication with the oligomerization reactor. The second-stage oligomerization charge stream is contacted with a oligomerization catalyst causing the unconverted ethylene from the oligomerization reactor to dimerize and trimerize while higher olefins also dimerize, trimerize and tetramerize to provide distillate range olefins. The second-stage oligomerization catalyst is a metal on a support catalyst, particularly Ni on a support. The second-stage oligomerization reactor process conditions may be selected to produce a higher percentage of jet range olefins which, when hydrogenated in a subsequent step result in a desirable jet-range hydrocarbon product. The predominance of the unconverted ethylene from the oligomerization reactor is dimerized, trimerized and tetramerized. In the two stages, typically all of the ethylene is converted. In the two-stage oligomerization process, the order of the 1^{st} and 2^{nd} stage catalysts can be reversed, i.e., the 1^{st} stage oligomerization catalyst is a metal on a support catalyst, and the 2^{nd} stage oligomerization catalyst is a zeolite.

The two stage oligomerization process conditions are selected for complete ethylene conversion. Under these conditions, the light paraffin, i.e., C₁-C₇ paraffins, production can be relatively high. This results in high carbon loss, and lower overall distillate yields. It is desirable to develop a process for oligomerizing ethylene that reduces carbon loss due to light paraffin production and that decreases the cost of acquiring and operating the oligomerization process using a single oligomerization reactor.

### BRIEF SUMMARY

This disclosure describes a process for producing a sustainable aviation fuel including oligomerizing a charge olefin stream over a single oligomerization catalyst to produce an oligomerized stream with greater than about 50% and less than about 99.5% olefins conversion per pass in the charge stream.

This disclosure also describes a process for producing a sustainable aviation fuel including oligomerizing a charge olefin stream over a single oligomerization catalyst at a maximum temperature of 275° C. to produce an oligomerized stream; and hydrogenating said oligomerized stream to provide a distillate stream containing fuel.

This disclosure also describes a process for producing a sustainable aviation fuel including oligomerizing a charge olefin stream over a single oligomerization catalyst at a maximum temperature of 275° C. to produce an oligomerized stream with greater than about 50% and less than about 99.5% olefins conversion per pass in the charge stream; and hydrogenating said oligomerized stream to provide a distillate stream containing fuel.

### BRIEF DESCRIPTION OF THE DRAWINGS

FIG. 1 is a flow scheme of a bio-olefin to SAF using a single-stage oligomerization process.

FIG. 2 is a flow scheme of a hydrogenation reactor.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

The term “bypass” means that the object is out of downstream communication with a bypassing subject at least to the extent of bypassing.

As used herein, the term “predominant” or “predominate” means greater than 50%, suitably greater than 75% and preferably greater than 90%.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripping columns may omit a reboiler at a bottom of the column and instead provide heating requirements and separation impetus from a fluidized inert media such as steam. Stripping columns typically feed a top tray and take main product from the bottom.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure. As used herein, the term “boiling point temperature” means atmospheric equivalent boiling point (AEBP) as calculated from the observed boiling temperature and the distillation pressure, as calculated using the equations furnished in ASTM D1160 appendix A7 entitled “Practice for Converting Observed Vapor Temperatures to Atmospheric Equivalent Temperatures”.

As used herein, the term “True Boiling Point” (TBP) means a test method for determining the boiling point of a material which corresponds to ASTM D-2892 for the production of a liquefied gas, distillate fractions, and residuum of standardized quality on which analytical data can be obtained, and the determination of yields of the above fractions by both mass and volume from which a graph of temperature versus mass % distilled is produced using fifteen theoretical plates in a column with a 5:1 reflux ratio.

As used herein, the term “T5”, “T90” or “T95” means the temperature at which 5 mass percent, 90 mass percent or 95 mass percent, as the case may be, respectively, of the sample boils using ASTM D-86 or TBP.

As used herein, the term “initial boiling point” (IBP) means the temperature at which the sample begins to boil using ASTM D-7184, ASTM D-86 or TBP, as the case may be.

As used herein, the term “end point” (EP) means the temperature at which the sample has all boiled off using ASTM D-7169, ASTM D-86 or TBP, as the case may be.

As used herein, the term “diesel” means hydrocarbons boiling in the range of an IBP between about 125° C. (257° F.) and about 175° C. (347° F.) or a T5 between about 150° C. (302° F.) and about 200° C. (392° F.) and the “diesel cut point” comprising a T95 between about 343° C. (650° F.) and about 399° C. (750° F.) using the TBP distillation method or a T90 between 280° C. (536° F.) and about 340° C. (644° F.) using ASTM D-86. The term “green diesel” means diesel comprising hydrocarbons not sourced from fossil fuels.

As used herein, the term “jet fuel” means hydrocarbons boiling in the range of a T10 between about 190° C. (374° F.) and about 215° C. (419° F.) and an end point of between about 290° C. (554° F.) and about 310° C. (590° F.). The term “green jet fuel” means jet fuel comprising hydrocarbons not sourced from fossil fuels.

The term “Cₓ” is to be understood to refer to molecules having the number of carbon atoms represented by the subscript “x”. Similarly, the term “Cₓ−” refers to molecules that contain less than or equal to x and preferably x and less carbon atoms. The term “Cₓ+” refers to molecules with more than or equal to x and preferably x and more carbon atoms.

### DETAILED DESCRIPTION

We have found that operating a single-stage oligomerization reactor under less severe conditions can result in a decrease in carbon loss due to the production of light paraffins, i.e., C₁-C₇. We found that it is possible to achieve a 90+% ethylene conversion per pass with a zeolite catalyst but this resulted in a carbon loss to light paraffins of 9% because the required temperature for 90+% ethylene conversion was very high. By targeting lower ethylene conversion per pass (with a zeolite or a metal on a zeolite catalyst) and by recovering the unconverted ethylene, it is possible to use less severe conditions and hence reduce the C loss. The catalyst can potentially be run at higher space velocity to control the olefinic hydrocarbon chain growth. Potentially, this catalyst in a single stage oligomerization makes on-spec diesel (ASTM D975) in addition to sustainable aviation fuel SAF. Additionally, a single stage oligomerization for olefins conversion to SAF (and renewable diesel) is less capitally and operationally intensive compared to a dual stage oligomerization process described below.

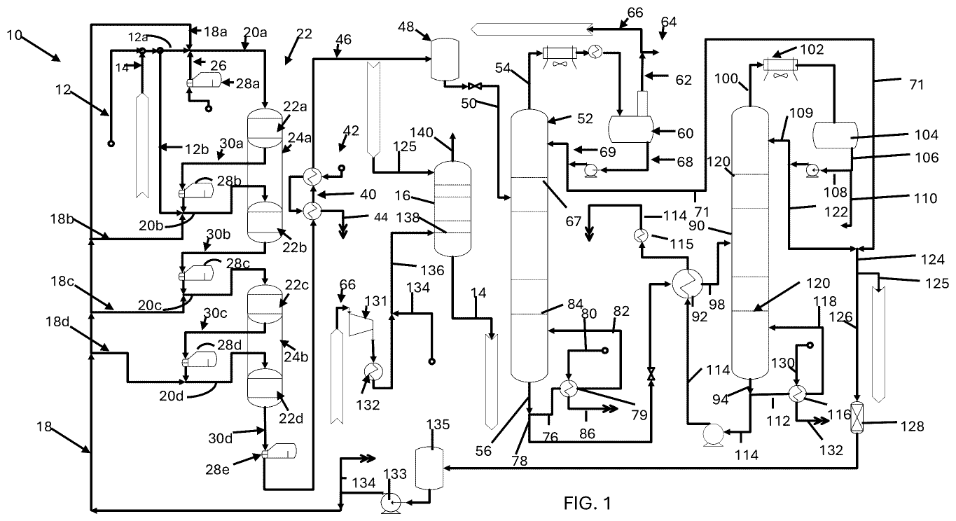

FIG. 1 shows a flow scheme 10 for oligomerizing a vapor olefin stream 12 followed by hydrogenation to make sustainable aviation fuel (SAF) and renewable diesel. The vapor olefin stream 12 may comprise substantial ethylene and propylene. The olefin stream may predominantly comprise ethylene and/or propylene. In an aspect, the vapor olefin stream may comprise at least 95 mol % ethylene and/or propylene. The vapor olefin stream in line 12 may be styled a light olefin stream. Additional olefinic species with carbon numbers ranging from C₄ to C₈ can be expected in the charge streams. The light olefin streams may be provided by the dehydration of ethanol or provided from a MTO unit. The light olefin stream may be at a temperature of about 20° C. (68° F.) to about 150° C. (302° F.) and a pressure of about 2.16 MPag (350 psig), preferably about 3.5 MPag (500 psig), to about 8.4 MPag (1200 psig).

The oligomerization reaction generates a large exotherm. For example, dimerization of ethylene can generate 612 kcal/kg (1100 BTU/lb) of heat. Consequently, this large exotherm must be managed. Accordingly, the light olefin stream in line 12 may be split into multiple olefin streams. In FIG. 1, the light olefin stream is split into two separate streams in lines 12a and 12b. More or less separate multiple olefin streams may be used. Up to six charge olefin streams are readily contemplated.

The olefin stream in line 12 is mixed with a recycle ethylene stream in line 14 from an ethylene sponge absorber column 16 and then may be split into equal aliquot multiple olefin streams in lines 12a and 12b. Alternatively, the vapor olefin stream in line 12 may be split into unequal streams. In an embodiment, the vapor olefin stream may be split into two streams of equal flow rates, each comprising 50 vol % of the charge olefin stream.

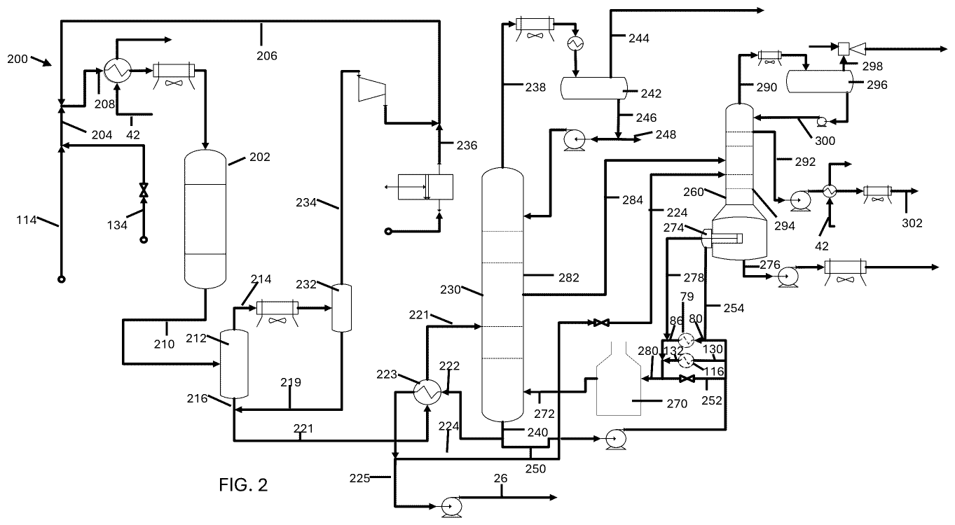

To manage the exotherm, the charge olefin stream 12 may be diluted with a recycle olefin stream in a recycle line 18 comprising C₄ to C₈ olefins and a recycle oil stream in line 26 from a hydrogenation unit (FIG. 2) to provide a first diluted charge olefin stream in line 20a. The first diluted charge olefin stream 20a may comprise no more than 50 wt % olefins, suitably no more than 30 wt % olefins and preferably no more than 20 wt % olefins. In an embodiment, the first diluted olefin stream 20a comprises about 10 to about 35 wt % C₂ to C₈ olefins. The first diluted olefin stream 20a may comprise no more than 50 wt % ethylene, suitably no more than 25 wt % ethylene and preferably no more than 20 wt % ethylene. In an embodiment, the first diluted charge olefin stream 20a comprises about 10 to about 20 wt % propylene. The first diluted charge olefin stream 20a may comprise no more than 50 wt % propylene, suitably no more than 25 wt % propylene and preferably no more than 20 wt % propylene. In an embodiment, the first diluted charge olefin stream 20a comprises about 10 to about 20 wt % propylene.

An oligomerization reactor 22 may comprise a series of oligomerization catalyst beds 22a, 22b, 22c and 22d in two oligomerization vessels 24a and 24b. Up to six oligomerization catalyst beds are readily contemplated. In FIG. 1, two oligomerization reactor vessels 24a and 24b are utilized. A parallel oligomerization reactor vessel may be used when catalyst beds in either the oligomerization reactor vessel 24a or 24b have deactivated during which the reactor beds in the reactor vessels are regenerated in situ by combustion of coke from the catalyst. The oligomerization reactor will be operated at a temperature from about 160° C. (320° F.) with greater than about 50% and no more than about 99.5% olefin, suitably no more than 99% olefin, more suitably no more than 98% olefin, even more suitably no more than 97% olefin, typically no more than 95% olefin, more typically no more than 90% olefin conversion per pass in the charge stream to about 275° C. (527° F.) and a pressure from about 2.06 MPa (gauge) (300 psig) to about 8.4 MPa (gauge) (1200 psig) and a weight hourly space velocity (WHSV) from about 0.5 h⁻¹ to about 10 h⁻¹ on an olefin basis. The oligomerization reactor will be operated to achieve an ethylene conversion of no more than about 99.5%, suitably no more than 99%, more suitably no more than 98%, even more suitably no more than 97%, typically no more than 95%, more typically no more than 90% per pass. By operating in these temperature, pressure, and WHSV ranges, which is less severe than necessary to achieve full ethylene conversion, a lower production of light paraffins is achieved and consequently a higher overall distillate (jet and diesel) yield. For example, the process generates less than about 10% light paraffins, more preferably less than about 5% light paraffins, more preferably below 3%, even more preferably below 2% and most preferably below 1%

The first diluted charge olefin stream in line 20a may be cooled by mixing with the recycle oil stream 26 that has been cooled in a first charge cooler 28a to provide a cooled diluted first charge olefin stream in line 20a and charged to the first bed 22a of the oligomerization reactor vessel 24a of the oligomerization reactor 22. The cooled diluted first charge olefin stream in line 20a may be charged at a temperature of about 180° C. (356° F.) to about 260° C. (500° F.) and a pressure of about 3.1 MPag (450 psig) to about 8.4 MPag (1200 psig). The charge cooler 28a may comprise a steam generator.

The diluted first charge olefin stream 20a may be charged to the first catalyst bed 22a in line 20a preferably in a down flow operation. However, upflow operation may be suitable. The cooled, diluted first charge olefin stream 20a is in a mixed vapor-liquid phase in which the vapor phase predominantly comprises ethylene. As oligomerization of ethylene, propylene and recycle olefins occurs in the first oligomerization catalyst bed 22a, an exotherm is generated due to the highly exothermic nature of the oligomerization reaction. Oligomerization of the first charge olefin stream 20a produces a first oligomerized stream in a first oligomerized line 30a at an elevated outlet temperature despite the cooling and dilution. The elevated outlet temperature is limited to between 150° C. (302° F.) and about 260° C. (500° F.).

The second charge olefin stream in line 12b may be mixed with the second recycle olefin stream in the second recycle olefin line 18b and with the first oligomerized stream in the first oligomerized line 30a to provide a mixed second charge olefin stream in line 20b. The first oligomerized stream in the first oligomerized line 30a may be cooled in a second charge cooler 28b which may be located externally to the oligomerization reactor 24a to provide a cooled second charge olefin stream in line 20b and charged to the second bed 22b of the oligomerization catalyst in the oligomerization reactor vessel 24a. The cooled second charge olefin stream 20b may comprise no more than 35 wt % C₂ to C₈ olefins, suitably no more than 25 wt % C₂ to C₈ olefins and preferably no more than 20 wt % C₂ to C₈ olefins. The cooled second charge olefin stream 20b may comprise no more than 30 wt % ethylene, suitably no more than 25 wt % ethylene and preferably no more than 20 wt % ethylene. The second charge olefin stream may comprise no more than 30 wt % propylene, suitably no more than 25 wt % propylene and preferably no more than 20 wt % propylene. The charge cooler 28b may comprise a steam generator.

The second cooled charge olefin stream in line 20b may be charged at a temperature of about 180° C. (356° F.) to about 230° C. (446° F.) and a pressure of about 3.1 MPag (450 psig) to about 8.4 MPag (1200 psig). The second cooled charge olefin stream will include diluent and olefins from the first oligomerized stream 30a. The diluted second cooled charge olefin stream in line 20b is in a mixed vapor-liquid phase in which the vapor phase predominantly comprises ethylene. The olefins from the first oligomerized stream 30a will oligomerize in the second catalyst bed 22b. Oligomerization of ethylene, propylene, recycle olefins and oligomers in the second olefin stream 30b in the second oligomerization catalyst bed 22b produces a second oligomerized olefin effluent stream in a second oligomerized line 30b at an elevated outlet temperature. The elevated outlet temperature may be limited to between 30° C. (54° F.) and about 50° C. (90° F.) above the inlet temperature to the catalyst bed 22b.

The second oligomerized stream in line 30b removed from the second oligomerization catalyst bed 22b in the first reactor vessel 24a may be mixed with the third recycle olefin stream in the third recycle olefin line 18c and with the second oligomerized stream in the second oligomerized line 30b to provide a mixed second charge olefin stream in line 20c. The second oligomerized stream in the second oligomerized line 30b may be cooled in a third charge cooler 28c which may be located externally to the oligomerization reactor vessel 24b to provide a cooled third charge olefin stream in line 20c and charged to the third bed 22c of the oligomerization catalyst in the oligomerization reactor vessel 24b. The third charge olefin stream in line 20c may comprise no more than 30 wt % ethylene, suitably no more than 25 wt % ethylene and preferably no more than 20 wt % ethylene. The third charge olefin stream may comprise no more than 30 wt % propylene, suitably no more than 25 wt % propylene and preferably no more than 20 wt % propylene. The third charge olefin stream may comprise no more than 30 wt % C₂ to C₈ olefins, suitably no more than 25 wt % C₂ to C₈ olefins and preferably no more than 20 wt % C₂ to C₈ olefins. The third charge cooler 28c may comprise a steam generator.

The third charge olefin stream in line 20c may be charged at a temperature of about 180° C. (356° F.) to about 230° C. (446° F.) and a pressure of about 3.1 MPag (450 psig) to about 8.4 MPag (1200 psig). The third charge olefin stream will include diluent and olefins from the second oligomerized olefin stream and the third recycle olefin stream. The olefins will oligomerize in the third catalyst bed 22c. Oligomerization of ethylene and propylene and oligomerization of oligomers in the third charge olefin stream in the third bed 22c of the oligomerization vessel 24b produces a third oligomerized stream in a third oligomerized line 30c at an elevated outlet temperature. In an embodiment, the third oligomerized stream is a penultimate oligomerized stream and the third oligomerized line 30c is a penultimate oligomerized line 30c. The elevated outlet temperature is limited to between 30° C. (54° F.) and about 50° C. (90° F.) above the inlet temperature to the catalyst bed 22c.

The third oligomerized stream in line 30c removed from the third oligomerization catalyst bed 22c in the second reactor vessel 24b may be mixed with the fourth recycle olefin stream in the fourth recycle olefin line 18d and with the third oligomerized stream in the third oligomerized line 30c to provide a mixed fourth charge olefin stream in line 20d. The third oligomerized stream in the third oligomerized line 30c may be cooled in a fourth charge cooler 28d which may be located externally to the second reactor vessel 24b to provide a cooled fourth charge olefin stream in line 20d and charged to the fourth bed 22d of the oligomerization catalyst in the second reactor vessel 24b. The third charge olefin stream in line 20d comprises no more than 35 wt % C₂ to C₈ olefins, suitably no more than 30 wt % C₂ to C₈ olefins and preferably no more than 25 wt % C₂ to C₈ olefins. The fourth charge olefin stream in line 20d may comprise no more than 30 wt % ethylene, suitably no more than 25 wt % ethylene and preferably no more than 20 wt % ethylene. The third charge olefin stream in line 20d may comprise no more than 30 wt % propylene, suitably no more than 25 wt % propylene and preferably no more than 20 wt % propylene. The charge cooler 18d may comprise a steam generator.

The cooled fourth recycle olefin charge stream in line 20d may be charged at a temperature of about 180° C. (356° F.) to about 230° C. (446° F.) and a pressure of about 3.1 MPag (450 psig) to about 8.4 MPa (g) (1200 psig). The cooled third recycle olefin charge stream in line 20d will include diluent and olefins from the third or penultimate oligomerized stream and C₄-C₈ olefins from the fourth recycle olefin stream 18d. The olefins will oligomerize over the fourth catalyst bed 22d. Oligomerization of ethylene and propylene in the fourth recycle olefin charge stream in the fourth bed 22d produces a fourth oligomerized stream in a fourth oligomerized line 30d at an elevated outlet temperature. The elevated outlet temperature is limited to between 30° C. (54° F.) and about 50° C. (90° F.) above the inlet temperature to the catalyst bed 22d.

The fourth oligomerized stream in line 30d exits the second reactor vessel 24b of the oligomerization reactor 22. In an embodiment, the fourth oligomerized stream in line 30d is a last oligomerized stream, and the fourth oligomerized line 30d is a last oligomerized line 30d.

The oligomerization reaction takes place predominantly in the liquid phase or in a mixed liquid and gas phase at a WHSV of 0.5 to 10 hr⁻¹ on an olefin basis. In a single pass through all the reactors of the same or similar catalyst in a stage, we can achieve over 90 to 95% conversion of ethylene. However, at over 90% ethylene conversion per pass through the stage over 10% yield of light paraffins results which represents a carbon loss. We have found that by reducing ethylene conversion per pass to below 90% per pass through the stage, light paraffin yield can be reduced to below to below 5%, suitably below 3% preferably below 2% and most preferably below 1%. However, by reducing conversion by reducing reaction temperature, or increasing WHSV, we can reduce ethylene conversion and thereby reduce light paraffin yield in a single stage pass. This is achieved without requiring a second stage of catalyst.

The oligomerization catalyst may include a zeolitic catalyst. The oligomerization catalyst may be considered a solid acid catalyst. The zeolite may comprise between about 5 and about 95 wt % of the catalyst, for example between about 5 and about 85 wt %. Suitable zeolites include zeolites having a structure from one of the following classes: MFI, MEL, ITH, IMF, TUN, FER, BEA, FAU, BPH, MEI, MSE, MWW, UZM-8, MOR, OFF, MTW, TON, MTT, AFO, ATO, and AEL. Three-letter codes indicating a zeotype are as defined by the Structure Commission of the International Zeolite Association and are maintained at http://www.iza-structure.org/databases. UZM-8 is as described in U.S. Pat. No. 6,756,030. In a preferred aspect, the oligomerization catalyst may comprise a zeolite with a framework having a ten-ring pore structure. Examples of suitable zeolites having a ten-ring pore structure include TON, MTT, MFI, MEL, AFO, AEL, EUO and FER. In a further preferred aspect, the oligomerization catalyst comprising a zeolite having a ten-ring pore structure may comprise a uni-dimensional pore structure. A uni-dimensional pore structure indicates zeolites containing non-intersecting pores that are substantially parallel to one of the axes of the crystal. The pores preferably extend through the zeolite crystal. Suitable examples of zeolites having a ten-ring uni-dimensional pore structure may include TON or MTT. In a further aspect, the oligomerization catalyst comprises an MTT zeolite.

The oligomerization catalyst may be formed by combining the zeolite with a binder, and then forming the catalyst into pellets. The pellets may optionally be treated with a phosphorus reagent to create a zeolite having a phosphorous component between 0.5 and 15 wt-% of the treated catalyst. The binder is used to confer hardness and strength on the catalyst. Binders include alumina, aluminum phosphate, silica, silica-alumina, zirconia, titania and combinations of these metal oxides, and other refractory oxides, and clays such as montmorillonite, kaolin, palygorskite, smectite and attapulgite. A preferred binder is an aluminum-based binder, such as alumina, aluminum phosphate, silica-alumina and clays.

One of the components of the catalyst binder utilized in the present invention is alumina. The alumina source may be any of the various hydrous aluminum oxides or alumina gels such as alpha-alumina monohydrate of the boehmite or pseudo-boehmite structure, alpha-alumina trihydrate of the gibbsite structure, beta-alumina trihydrate of the bayerite structure, and the like. A suitable alumina is available from UOP LLC under the trademark VERSAL. A preferred alumina is available from Sasol North America Alumina Product Group under the trademark CATAPAL. This material is an extremely high purity alpha-alumina monohydrate (pseudo-boehmite) which after calcination at a high temperature has been shown to yield a high purity gamma-alumina.

A suitable oligomerization catalyst is prepared by mixing proportionate volumes of zeolite and alumina to achieve the desired zeolite-to-alumina ratio. In an embodiment, the zeolite content may about 5 to about 85, for example about 20 to about 82 wt % zeolite, and the balance alumina powder will provide a suitably supported catalyst. A silica support is also contemplated.

Monoprotic acid such as nitric acid or formic acid may be added to the mixture in aqueous solution to peptize the alumina in the binder. Additional water may be added to the mixture to provide sufficient wetness to constitute a dough with sufficient consistency to be extruded or spray dried. Extrusion aids such as cellulose ether powders can also be added. A preferred extrusion aid is available from The Dow Chemical Company under the trademark Methocel.

The paste or dough may be prepared in the form of shaped particulates, with the preferred method being to extrude the dough through a die having openings therein of desired size and shape, after which the extruded matter is broken into extrudates of desired length and dried. A further step of calcination may be employed to give added strength to the extrudate. Generally, calcination is conducted in a stream of air at a temperature from about 260° C. (500° F.) to about 815° C. (1500° F.). The MTT catalyst is not selectivated to neutralize acid sites such as with an amine.

The extruded particles may have any suitable cross-sectional shape, i.e., symmetrical or asymmetrical, but most often have a symmetrical cross-sectional shape, preferably a spherical, cylindrical or polylobal shape. The cross-sectional diameter of the particles may be as small as 40 μm; however, it is usually about 0.635 mm ( 1/40 inch) to about 12.7 mm (½ inch), preferably about 0.79 mm ( 1/32 inch) to about 6.35 mm (¼ inch), and most preferably about 0.06 mm ( 1/24 inch) to about 4.23 mm (⅙ inch).

In one exemplary embodiment, a zeolite catalyst disposed on a high purity pseudo boehmite alumina substrate in a ratio of about 90/10 to about 20/80 and preferably between about 20/80 and about 50/50 is provided in a catalyst bed or more in the oligomerization reactor 22. Optionally, all or a portion of the alumina can be replaced by amorphous silica alumina.

The zeolite catalyst is advantageous as an oligomerization catalyst. The zeolitic catalyst has relatively low sensitivity towards oxygenates contamination. Consequently, a smaller degree of removal of oxygenates is required of olefinic feed in line 12 if produced from an alcohol dehydration process.

In another aspect, the oligomerization zeolite catalyst can include a metal from either Group VIII and/or Group VIB in the periodic table using Chemical Abstracts Service notations. In an aspect, the catalyst has a Group VIII metal promoted with a Group VIB metal. The Group VIII metal, preferably nickel, should be present in a concentration of about 0.2 to about 15 wt-% and the Group VIB metal, preferably tungsten, should be present in a concentration of about 0 to about 12 wt-%.

One of preferred oligomerization catalysts is impregnated with about 0.2 to about 15 wt-% nickel in the form of 3.175 mm (0.125 inch) extrudates and an apparent density of about 0.45 to about 0.65 g/ml. It is also contemplated that metals can be incorporated onto the support by other methods such as ion-exchange and co-mulling.

One of preferred metal-zeolite oligomerization catalysts may be prepared by loading metals directly on zeolite by impregnation, ion-exchange, or co-mulling methods The metal-loaded zeolites may be further formed by themselves or with a binder.

One of preferred metal-zeolite oligomerization catalysts may be prepared by loading metals directly on binder by impregnation, ion-exchange, or co-mulling methods The metal-loaded binder may be further formed with zeolites.

The oligomerization catalyst can be regenerated upon deactivation. Suitable regeneration conditions include subjecting the catalyst, for example, in situ, to hot air at about 400° C. to about 500° C. To facilitate regeneration without downtime, a swing bed arrangement may be employed with an alternative oligomerization reactor. The regeneration gas may comprise air with an increased or decreased concentration of oxygen. Activity and selectivity of the regenerated catalyst is comparable to fresh catalyst.

These oligomerization reactions are also exothermic in nature. The last oligomerized olefin stream in line 24d includes the diluent stream from diluent line 14 added to the first charge olefin stream in the first charge olefin line 12a and carried through the first-stage oligomerization catalyst beds 22a-22d. The diluent stream is then transported into the second-stage oligomerization reactor 32 in line 28 to absorb the exotherm in the second-stage oligomerization reactor. A dedicated diluent line to the second-stage oligomerization reactor 32 is also contemplated for prompt control of exotherm rise or to cool down the second-stage oligomerization reactor 32.

The last oligomerized stream in the last oligomerized line 30d has an increased concentration of ethylene and propylene oligomers compared to the light olefin stream in line 12. The last oligomerized stream in line 30d is cooled by steam generation in a steam generator 28e.

The last oligomerized stream 30d is heat exchanged in a heat exchanger 40 with cold ethanol in a stream 42. After exiting the heat exchanger 40, the heated ethanol is conveyed in a stream through line 44 to an ethanol dehydration unit (not shown). The cooled oligomer stream is conveyed through line 46 to a holding tank 48 and then through line 50 to a dealkanizer column 52. The oligomerized olefin stream in line 46 is at a temperature from about 160° C. (320° F.) to about 190° C. (374° F.) and a pressure of about 3.9 MPa (gauge) (550 psig) to about 7 MPa (gauge) (1000 psig). We have found that light alkanes such as ethane and/or propane are generated in the oligomerization reactor 22 which must be removed for fuels production particularly to facilitate light olefin recycle to the oligomerization reactor 22. Light alkanes are inert and would accumulate in the recycle loop. Hence, the oligomerized stream in line 30d is dealkanized by fractionation in a dealkanizer column 52 to provide a light alkane stream and a dealkanized stream. In an embodiment, the light alkane stream is an ethane stream in which case the dealkanizer column 52 is a deethanizer column. In another embodiment, the light alkane stream is a propane stream in which case the dealkanizer column 52 is a depropanizer column. The light alkane stream may contain ethane and/or propane and can also be a mixture of ethane and propane.

In the dealkanizer column 52, light alkanes such as C3- and suitably C2-hydrocarbons, are separated perhaps in a light alkane overhead stream in an overhead line 54 from perhaps a dealkanized bottoms stream in a bottoms line 56 comprising C4+ and suitably C3+ hydrocarbons. The dealkanizer column 52 may be operated at a bottoms temperature of about 177° C. (350° F.) to about 302° C. (575° F.) and an overhead pressure of about 207 kPa (gauge) (30 psig) to about 965 kPa (gauge) (140 psig) if operated as a deethanizer column. The dealkanizer column 52 may be operated at a bottoms temperature of about 194° C. (381° F.) to about 333° C. (630° F.) and an overhead pressure of about 207 kPa (gauge) (30 psig) to about 1.38 MPa (gauge) (200 psig) if operated as a depropanizer column.

The light alkane overhead stream in the overhead line 54 may be cooled and separated in a dealkanizer receiver 60 to provide a dealkanized off-gas stream in an off-gas line 62 which splits into lines 64 and 66. The stream in line 64 is directed to an off-gas fuel knockout drum to be taken as fuel gas and the stream in line 66 is recycled to the ethylene absorber column 16. The bottoms stream in line 68 is split into two streams in lines 69 and 71. The stream in line 69 is returned to the dealkanizer column above a top deck 67. The stream in line 71 is mixed with the stream in line 122.

The net dealkanized stream in the net bottoms dealkanizer line 56 is split into two lines 76 and 78. The stream in line 76 is reboiled in a heat exchanger 79 by heat exchange with a stripper bottoms stream in line 80. The boiling dealkanized stream is returned to the dealkanizer column 52 through line 82 and enters the dealkanizer column 52 below a bottom deck 84. The cooled stripper bottoms stream in line 86 is sent back to the stripper reboiler in the hydrogenation section.

The dealkanized stream in line 78 is directed to an olefin splitter column 90 through a heat exchanger 92 where it is cooled in exchange with an oligomerization splitter column bottoms stream in line 114. The heated bottoms stream is conveyed through line 114 through a heat exchanger 115 and sent to a hydrogenation unit for hydrogenation. The cooled dealkanized stream in line 98 enters the olefin splitter column 90 near a midpoint of its height dimension. The dealkanized stream is split by fractionation in the olefin splitter column 90 into a light olefin stream perhaps in an olefin splitter overhead line 100 and a heavy oligomerized stream perhaps in the olefin splitter bottoms line 94. The olefin splitter overhead stream may be chilled to about 19° C. (66° F.) to about 93° C. (200° F.) in a chiller 102 and fully condensed in a receiver 104 to provide an olefin split condensate stream in line 106. The light olefin condensate from a bottom of the olefin splitter receiver in line 106 may be refluxed stream in line 108 and a light olefin drag stream in a recycle line 110 recycled to a hydrogenation reactor in FIG. 2. The light olefin stream in line 108 is split into a reflux stream 109 which is refluxed back to the column in line 109 and enters the splitter column 90 above a top deck 120 and a light olefin stream in line 122 is mixed with the stream in line 71 to form a combined stream in line 124. The combined stream in line 124 is split into a first stream 125 that is directed to the ethylene sponge absorber column 16 and a second stream in line 126. The second stream in line 126 is conveyed through a dryer 128 and then a holding tank 135. A pump 133 pumps the C₄ to C₈ olefins in a stream in a first line 134 to a hydrogenation unit in FIG. 2 and in a second line 18 that is recycled to the oligomerization reactor 22.

The heavy oligomerized stream in the splitter bottoms line 94 may be split between a reboil stream in a splitter reboil line 112 and a heavy oligomerized stream in a net splitter bottoms line 114. The splitter reboil stream in the net splitter bottoms line 112 is reboiled by heat exchange in an olefin splitter reboiler 116 with a stripper bottoms stream in line 130. A reboiling splitter bottoms stream emerges from the reboiler 116 in line 118 and is returned to the splitter column 90 below a bottom deck 120 while a cooled stripper bottoms stream in line 132 is returned to the splitter bottoms in line 132. The heavy oligomerized stream in line 114 comprises C9+ olefins that once cooled in the heat exchanger 92 is conveyed through line 96 to a hydrogenation section in FIG. 2.

The heavy oligomerized stream in the net olefin splitter bottoms line 94 comprising distillate-range C9+ oligomerized olefins may be hydrogenated to saturate the olefinic bonds in a hydrogenation reactor to provide a distillate fuel stream. This step is performed to ensure the product fuel meets or exceeds the thermal oxidation requirements specified in ASTM D7566-20 for Alcohol to Jet Synthesized Paraffinic Kerosene (ATJ-SPK). The diesel can qualify for AST D975 No. 2-D grade diesel which may be known as Renewable Diesel. Additionally, hydrogenating the heavy oligomerized stream will provide the paraffin stream that may be used as the diluent stream in line 26.

The ethylene sponge absorber column 16 receives a stream from the dealkanizer column 52 in line 136 which is compressed with compressor 131 through heat exchanger 132 where it is cooled and then mixed with an ethylene stripper off-gas stream in line 134, and the combined stream in line 136 enters the sponge absorber column at a first deck 138. The sponge absorber column 16 also receives a stream from the olefin splitter column 90 in the line 125 taken from the combined stream in line 124 comprising C₄-C₈ olefins. The olefin stream in line 125 is counter-currently contacted with the dealkanizer off-gas stream to absorb ethylene into the olefin stream. An overhead vapor stream of ethylene in line 140 is produced and directed to further processing such as in an off-gas knockout drum. The bottoms stream enriched in ethylene is conveyed through line 14 to the oligomerization reactor 22. The sponge absorber column is operated at a bottoms temperature of about 50° C. (122° F.) to about 135° C. (275° F.) and an overhead pressure of about 2413 kPa (gauge) (350 psig) to about 3103 kPa (gauge) (450 psig). Turning to the hydrogenation unit 200 in FIG. 2, the heavy oligomerized stream in the net olefin splitter bottoms line 114 from FIG. 1 comprising distillate-range C9+ oligomerized olefins may be hydrogenated to saturate the olefinic bonds in a hydrogenation reactor 202 to provide a distillate fuel stream. This step is performed to ensure the product fuel meets or exceeds the thermal oxidation requirements specified in ASTM D7566-20 for Alcohol to Jet Synthesized Paraffinic Kerosene (ATJ-SPK). The diesel can qualify for AST D975 No. 2-D grade diesel which may be known as Renewable Diesel. Additionally, hydrogenating the heavy oligomerized stream will provide the paraffin stream that may be used as the diluent stream in line 26. The heavy oligomerized stream in line 114 may be combined with the light olefin liquid stream comprising C₂ to C₈ olefins in line 134 also from FIG. 1 to produce a combined olefin stream in line 204. The combined olefin stream in line 202 may also be combined with a hydrogen stream in line 206 to provide a combined hydrogenation charge stream in line 208 which is cooled perhaps by heat exchange with a feed ethanol stream and charged to the hydrogenation reactor 202 at 125° C. (257° F.) to about 204° C. (400° F.) and 2.8 MPa (400 psig) to about 6.9 MPa (1000 psig). An excess of hydrogen may be employed to ensure complete saturation such as about 1.5 to about 5.0 of stochiometric hydrogen.

Hydrogenation is typically performed using a conventional hydrogenation or hydrotreating catalyst, and can include metallic catalysts containing, e.g., palladium, rhodium, nickel, ruthenium, platinum, rhenium, cobalt, molybdenum, or combinations thereof, and the supported versions thereof. Catalyst supports can be any solid, inert substance including, but not limited to, oxides such as silica, alumina, titania, calcium carbonate, barium sulfate, and carbons. The catalyst support can be in the form of powder, granules, pellets, or the like.

In an exemplary embodiment, hydrogenation is performed in the hydrogenation reactor 202 that includes a platinum-on-alumina catalyst, for example about 0.1 wt % to about 2 wt %, preferably about 0.5 wt % to about 0.9 wt %, platinum-on-alumina catalyst. In another embodiment, the hydrogenation catalyst comprises about 5 to about 30 wt % nickel catalyst. The hydrogenation reactor 80 converts the olefins into a paraffin product having the same carbon number distribution as the olefins, thereby forming distillate-range paraffins suitable for use as jet and diesel fuel.

The hydrogenated distillate stream discharged from the hydrogenation reactor 202 in line 210 may be separated in a hot separator 212 which provides a hydrocarbon split. In the hot separator 212, the hydrogenated distillate stream is separated into a hot hydrogenated vapor stream in an overhead line 214 and a hot distillate liquid stream in the hot separator bottoms line 216. The hot separator may be operated at a temperature of about 204° C. (400° F.) to about 343° C. (650° F.) and a pressure of 2.8 MPa (400 psig) to about 6.9 MPa (1000 psig).

The hydrogenated distillate liquid stream in the bottoms line 216 may be combined with a cold heavy distillate liquid stream in a cold bottoms line 219 to provide a combined separator liquid distillate stream in line 221. The combined separator liquid distillate stream in the combined bottoms line 221 may be heated by heat exchange with a stripped stream in line 222 in a stripping heat exchanger 223. The combined bottoms line 221 may be stripped of volatiles in a stripping column 230. The stripped stream in line 222 is split into streams in line 224 and 225. The stream in line 224 is charged to a jet fractionation column 260 and the stream in line 225 is pumped through line 26 to the oligomerization section 22.

The hot vapor distillate stream in the hot overhead line 214 may be cooled and fed to a cold separator 232. The cold separator 232 separates the cooled hot vapor hydrogenated stream in the hot overhead line 214 into a cold vapor hydrogenated stream in a cold overhead line 234 and the cold heavy liquid distillate stream in a cold bottoms line 219. The cold vapor hydrogenated stream in the cold overhead line 234 may be compressed and combined with make-up hydrogen stream in line 236 to provide the hydrogen stream in line 206. The cold liquid distillate stream in the bottoms line 219 may be combined with the hot liquid distillate stream in the hot separator bottoms line 216 to provide the combined separator liquid distillate stream in the combined bottoms line 221 and fed to the stripping column 230. The combined separator liquid distillate stream in the combined bottoms line 221 may be heated by heat exchange with a stripped stream in line 222 in a stripping heat exchanger 223. The cold separator 232 may be operated at a temperature of about 32° C. (90° F.) to about 71° C. (150° F.) and a pressure of about 2.8 MPa (400 psig) to about 4.5 MPa (650 psig).

The stripping column 230 may involve stripping with a reboiler to remove naphtha and lighter materials from the combined separator liquid distillate stream in the combined bottoms line 221. The stripping column 230 removes residual light gases from the liquid distillate streams to provide a stripping overhead stream in a stripping overhead line 238 and a stripped distillate stream in a stripping bottoms line 240. The stripping overhead stream in the stripping overhead line 238 is cooled and separated in a stripping receiver 242 to provide a stripping off-gas stream in a stripping receiver overhead line 244 and a condensate stream in line 246 which is refluxed to the stripping column 230. The stripping off-gas stream in line 244 may be transported to the fuel gas header. The stripping off gas stream in the receiver overhead line 244 can be used to fuel the stripper reboiler 270 for the stripping column 282.

A reflux stream taken from the condensate stream in line 246 may be refluxed to the stripping column 230 while a wild naphtha stream is taken in line 248 from the condensate stream. The stripping column 230 may be operated at a bottoms temperature of about 232° C. (450° F.) to about 388° C. (730° F.), preferably no more than 360° C. (680° F.), and an overhead pressure of about 207 kPa (30 psig) to about 1380 kPa (200 psig).

After undergoing stripping to remove volatiles in the stripping column 230, the stripped distillate stream in the stripping bottoms line 240 comprises C9+ materials. The stripped distillate stream in the stripping bottoms line 240 may be split into a diluent stream in line 222 and stripping reboil stream in line 250. The diluent stream is taken in line 222 is heat exchanged with the combined separator liquid distillate stream in the combined bottoms line 221 in the heat exchanger 223. The diluent effluent from heat exchanger 223 is split into two streams, 224 and 225. Stream 225 is pumped through recycle oil pump and recycled to the oligomerization section 22 as stream 26 to absorb the exotherm in FIG. 1. The diluent stream in line 26 may be recycled back to be mixed with the charge olefin streams in lines 12a and 12b in the oligomerization section 22 in FIG. 1, preferably the first charge olefin stream in line 12a, to provide the first diluted olefin charge stream in line 18a to absorb the exotherm in the oligomerization reactor 22. The stripper bottom stream in the diluent line 222 is paraffinic, so it will be inert to the oligomerization and hydrogenation reactions to which it may be subject. The stripping reboil stream in line 250 is pumped and split into a net stripped stream in line 252, and a jet fractionation reboiling stream in line 254. The net stripped stream in line 252 is fed to a stripper reboiler 270 and then in a stream through line 272 into the stripper column 230 near the bottoms. In an embodiment, the jet fractionation reboiling stream in line 254 may be fed to a stabbed-in reboiler 274 to reboil the jet fractionation column 260 and provide a jet fractionator bottoms stream 276. The stream in line 276 is pumped through a cooler and collected as diesel blend stock. The hot oil stream in line 80 is forwarded to the dealkaziner reboiler 79 in FIG. 1 to reboil the splitter reboil stream in the splitter reboil line 76 by heat exchange in the olefins splitter reboiler 79. The heated stream from the heat exchanger in line 82 is then returned to the dealkanizer column 67. The cooled hot oil stream from the heat exchanger in line 86 is charged to the stripper reboiler 270. The stream in line 118 is returned to the olefin splitter column 90.

The cooled jet fractionation reboiling stream in line 278 may mixed with the stream in line 252 to form a combined input stream in line 280 to the stripper reboiler and reboiled in the stripper reboiler 270 which may be a fired heater. The reboiled stripped stream in line 272 is returned to a bottom of the stripping column 230.

An intermediate stream comprising C₈+ hydrocarbons may be taken from a side 282 of the stripping column 230 in line 284. The intermediate stream typically comprises C₈-C₉ hydrocarbons. This intermediate steam is taken to prevent C₈-C₉ paraffins from separating in the stripped bottoms stream and recycling as diluent oil to the oligomerization section 22. In the oligomerization section 22, the C₈ and C₉ paraffins would go into the overhead of the olefins splitter column and recycle to the oligomerization reactors 22 with no way of exiting the oligomerization section 22 since they are inert to oligomerization. Hence, the intermediate stream is taken in line 284 from the side of the stripping column and fed to the jet fractionation column 260.

In the jet fractionation column 260, the stripped bottoms stream 224 and the intermediate stripped stream may be separated into a jet overhead stream in an overhead line 290, an intermediate synthetic aviation fuel (SAF) stream boiling in the jet fuel range in a side line 292 from a side 294 of the jet fractionation column 260 and a heavy diesel stream in a jet bottoms line 276. The jet fractionation column 260 may be operated at a bottoms temperature of about 288° C. (550° F.) to about 343° C. (650° F.) and an overhead absolute pressure of about 117 kPa (17 psia) to about 207 kPa (30 psia).

The jet fractionation overhead stream in the overhead line 290 may be cooled, condensed and fed to a jet fractionation receiver 296. Non-condensables can be taken in a jet receiver overhead line 298. A jet fractionation overhead condensate stream may be refluxed back to the jet fractionation column 260 in a jet fractionator overhead liquid line 300.

The SAF stream taken in the side line 292 comprises kerosene range C₈-C₁₈ hydrocarbons which may be cooled and taken as a jet fuel product stream meeting applicable SPK standards. In an alternative embodiment, the green jet stream may be taken from the condensate stream in line 300 from the jet fractionation receiver 296 instead of refluxing all of the condensate to the column. This green jet stream taken from line 300 may have to be further stripped to remove light ends. In such an embodiment, no side line 292 would be taken to recover the green jet fuel stream. A green jet fuel stream comprising SAF meeting applicable qualifying standards is taken in SAF product line 302.

### Example

Yields of oligomerization of ethylene are estimated using a single oligomerization reactor to produce SAF and renewable diesel. The reaction conditions are shown in Table 1 and the yields are shown in Table 2.

TABLE 1

| | | Top | Reactor | | WH SV | WH | Max | Mass |
|---|---|---|---|---|---|---|---|---|
| | | Bed | outlet | | FF | SV FF | Top | Balance |
| | Starting | Heater | pressure | P:O | (Olefins, | (Total, | Bed | by |
| Condition | HOS | Temp. | (psig) | (w/w) | hr − 1) | hr − 1) | Temp | Controller |
| 1 | 0 | 235 | 900 | 4 | 0.5 | 2.52 | 235.7 | 99.43 |
| 2 | 14 | 215 | 900 | 4 | 0.5 | 2.51 | 213.7 | 97.99 |
| 3 | 58 | 215 | 900 | 4 | 0.5 | 2.52 | 213.1 | 100.89 |
| 4 | 108 | 215 | 900 | 1.5 | 0.5 | 1.26 | 217.3 | 99.5 |
| 5 | 147 | 215 | 500 | 1.5 | 0.5 | 1.26 | 214 | 99.01 |

TABLE 2

| | | C₁-C₃ | C₄-C₇ | C₄-C₇ | | |
|---|---|---|---|---|---|---|
| | C₂ = | Yield | Paraffins | Olefins | C₈-C₁₇ | C₁₈ |
| Condition | Conversion | Total | Yield | Yield | Yield | Yield |
| 1 | 97.08 | 4.2 | 10.5 | 18.4 | 60.9 | 3.11 |
| 2 | 53.34 | 1.2 | 1.3 | 4.8 | 43.5 | 2.56 |
| 3 | 19.25 | 0.6 | 0.5 | 2 | 15.2 | 0.96 |
| 4 | 94.3 | 2.9 | 5.8 | 17.6 | 64.1 | 3.8 |
| 5 | 23.87 | 0.4 | 0.4 | 1.9 | 19.2 | 1.96 |

It is possible to achieve 90+% ethylene conversion per pass with a TON zeolite catalyst described herein. However, the C loss to light paraffins is high (>9%) because the max bottom temperature is high.

By targeting lower ethylene conversion per pass and by recovering the unconverted ethylene, it is possible to use less severe conditions and hence reduce the C loss. The catalyst can potentially be run at higher space velocity to control the olefinic hydrocarbon chain growth.

### Specific Embodiments

While the following is described in conjunction with specific embodiments, it will be understood that this description is intended to illustrate and not limit the scope of the preceding description and the appended claims.

A first embodiment of the invention is a process for producing a sustainable aviation fuel comprising oligomerizing a charge olefin stream over a single oligomerization catalyst to produce an oligomerized stream with greater than about 50% and less than about 99.5% olefins conversion per pass in the charge stream. An embodiment of the invention is one, any or all of prior embodiments in this paragraph up through the first embodiment in this paragraph wherein the charge olefin stream is obtained by dehydration of ethanol. An embodiment of the invention is one, any or all of prior embodiments in this paragraph up through the first embodiment in this paragraph wherein the charge olefin stream is obtained from a methanol to olefin unit. An embodiment of the invention is one, any or all of prior embodiments in this paragraph up through the first embodiment in this paragraph wherein the oligomerizing step further comprises oligomerizing the charge olefin stream in an oligomerization reactor over a catalyst bed of a zeolite catalyst or a metal on a zeolite catalyst. An embodiment of the invention is one, any or all of prior embodiments in this paragraph up through the first embodiment in this paragraph wherein the oligomerized stream is dealkanized by fractionation in a dealkanizer column to provide a light alkane stream and a dealkanized stream. An embodiment of the invention is one, any or all of prior embodiments in this paragraph up through the first embodiment in this paragraph wherein the dealkanized stream is split by fractionation in an olefin splitter column into a light olefin stream in an olefin splitter overhead line and a heavy oligomerized stream in an olefin splitter bottoms line. An embodiment of the invention is one, any or all of prior embodiments in this paragraph up through the first embodiment in this paragraph wherein the olefin splitter overhead stream may be chilled to about 19° C. (66° F.) to about 93° C. (200° F.) in a chiller and fully condensed in a knockout drum to provide an olefin split condensate stream. An embodiment of the invention is one, any or all of prior embodiments in this paragraph up through the first embodiment in this paragraph wherein the light alkane stream comprises C2- or C3-hydrocarbons. An embodiment of the invention is one, any or all of prior embodiments in this paragraph up through the first embodiment in this paragraph wherein the light alkane stream is charged to a sponge absorber column. An embodiment of the invention is one, any or all of prior embodiments in this paragraph up through the first embodiment in this paragraph wherein the light olefin stream from the olefin splitter column is charged to the sponge absorber column above a top plate of the sponge absorber. An embodiment of the invention is one, any or all of prior embodiments in this paragraph up through the first embodiment in this paragraph wherein the light olefin stream counter currently contacts the light alkane stream in the sponge absorber column.

A second embodiment of the invention is a process for producing a sustainable aviation fuel comprising oligomerizing a charge olefin stream over a single oligomerization catalyst at a maximum temperature of 275° C. to produce an oligomerized stream; and hydrogenating the oligomerized stream to provide a distillate stream containing fuel. An embodiment of the invention is one, any or all of prior embodiments in this paragraph up through the second embodiment in this paragraph wherein the charge olefin stream is obtained by dehydration of ethanol. An embodiment of the invention is one, any or all of prior embodiments in this paragraph up through the second embodiment in this paragraph wherein the charge olefin stream is obtained from a methanol to olefin unit. An embodiment of the invention is one, any or all of prior embodiments in this paragraph up through the second embodiment in this paragraph wherein the oligomerizing step further comprises oligomerizing the charge olefin stream in an oligomerization reactor over a catalyst bed of a zeolite catalyst or a metal on a zeolite catalyst. An embodiment of the invention is one, any or all of prior embodiments in this paragraph up through the second embodiment in this paragraph wherein the light olefin stream counter currently contacts the light alkane stream in a sponge absorber column.

A third embodiment of the invention is a process for producing a sustainable aviation fuel comprising oligomerizing the charge olefin stream over a single oligomerization catalyst at a maximum temperature of 275° C. to produce an oligomerized stream with greater than about 50% and less than about 99.5% olefins conversion per pass in the charge stream; and hydrogenating the oligomerized stream to provide a distillate stream containing the fuel. An embodiment of the invention is one, any or all of prior embodiments in this paragraph up through the third embodiment in this paragraph wherein the oligomerizing step further comprises oligomerizing the charge olefin stream in an oligomerization reactor over a catalyst bed of a zeolite catalyst or a metal on a zeolite catalyst. An embodiment of the invention is one, any or all of prior embodiments in this paragraph up through the third embodiment in this paragraph wherein the light olefin stream counter currently contacts the light alkane stream in a sponge absorber column. An embodiment of the invention is one, any or all of prior embodiments in this paragraph up through the third embodiment in this paragraph wherein a bottoms stream of the sponge absorber column is mixed with the charge olefin stream.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for producing a sustainable aviation fuel comprising:
oligomerizing a charge olefin stream over a single oligomerization catalyst to produce an oligomerized stream with greater than about 50% and less than about 99.5% olefins conversion per pass in the charge stream.

2. The process of claim 1 wherein the charge olefin stream is obtained by dehydration of ethanol.

3. The process of claim 1 wherein the charge olefin stream is obtained from a methanol to olefin unit.

4. The process of claim 1 wherein said oligomerizing step further comprises oligomerizing said charge olefin stream in an oligomerization reactor over a catalyst bed of a zeolite catalyst or a metal on a zeolite catalyst.

5. The process of claim 1 wherein the oligomerized stream is dealkanized by fractionation in a dealkanizer column to provide a light alkane stream and a dealkanized stream.

6. The process of claim 5 wherein the dealkanized stream is split by fractionation in an olefin splitter column into a light olefin stream in an olefin splitter overhead line and a heavy oligomerized stream in an olefin splitter bottoms line.

7. The process of claim 6 wherein the olefin splitter overhead stream may be chilled to about 19° C. (66° F.) to about 93° C. (200° F.) in a chiller and fully condensed in a knockout drum to provide an olefin split condensate stream.

8. The process of claim 6 wherein the light alkane stream comprises C2- or C3-hydrocarbons.

9. The process of claim 8 wherein the light alkane stream is charged to a sponge absorber column.

10. The process of claim 9 wherein the light olefin stream from the olefin splitter column is charged to the sponge absorber column above a top plate of the sponge absorber.

11. The process of claim 10 wherein the light olefin stream counter currently contacts the light alkane stream in the sponge absorber column.

12. A process for producing a sustainable aviation fuel comprising:
oligomerizing a charge olefin stream over a single oligomerization catalyst at a maximum temperature of 275° C. to produce an oligomerized stream; and
hydrogenating said oligomerized stream to provide a distillate stream containing fuel.

13. The process of claim 12 wherein the charge olefin stream is obtained by dehydration of ethanol.

14. The process of claim 12 wherein the charge olefin stream is obtained from a methanol to olefin unit.

15. The process of claim 12 wherein said oligomerizing step further comprises oligomerizing said charge olefin stream in an oligomerization reactor over a catalyst bed of a zeolite catalyst or a metal on a zeolite catalyst.

16. The process of claim 12 wherein the light olefin stream counter currently contacts the light alkane stream in a sponge absorber column.

17. A process for producing a sustainable aviation fuel comprising:
oligomerizing the charge olefin stream over a single oligomerization catalyst at a maximum temperature of 275° C. to produce an oligomerized stream with greater than about 50% and less than about 99.5% olefins conversion per pass in the charge stream; and
hydrogenating said oligomerized stream to provide a distillate stream containing the fuel.

18. The process of claim 17 wherein said oligomerizing step further comprises oligomerizing said charge olefin stream in an oligomerization reactor over a catalyst bed of a zeolite catalyst or a metal on a zeolite catalyst.

19. The process of claim 17 wherein the light olefin stream counter currently contacts the light alkane stream in a sponge absorber column.

20. The process of claim 19 wherein a bottoms stream of the sponge absorber column is mixed with the charge olefin stream.
